SEQUENCE LISTING (28 sequences)

SEQ ID NO: 1 - DNA, 197 bp, synthetic construct
  source 1..197: mol_type = other DNA
aatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattgcgtca

SEQ ID NO: 2 - DNA, 112 bp, synthetic construct
  source 1..112: mol_type = other DNA
cagagatgttttcttgggttaattgaggcctgagtataaggtgacttatacttgtaatctatctaaacggggaacctctctagtagacaatcccgtgctaaattgtaggact

SEQ ID NO: 3 - DNA, 182 bp, synthetic construct
  source 1..182: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccaccccca

SEQ ID NO: 4 - DNA, 131 bp, synthetic construct
  source 1..131: mol_type = other DNA
agacgctacggacttaaataattgagccttaaagaagaaattctttaagtggatgctctcaaactcagggaaacctaaatctagttatagacaaggcaatcctgagccaagccgaagtagtaattagtaag

SEQ ID NO: 5 - DNA, 209 bp, synthetic construct
  source 1..209: mol_type = other DNA
gtagcgggcacccgtcgtaacgcgcggtagggcgtcggtcccccctcccacggcggaggggaggcttaaggtacgtgctaaacccccagcgatggggcctgtaggaaaagccctggtacggcgaagcctacggggaccgcccgatggcggtcggatgccggcgggccacggagcccccggcgtcgattgctgttatgcccttagatgtt

SEQ ID NO: 6 - DNA, 224 bp, synthetic construct
  source 1..224: mol_type = other DNA
agtttgaggcaataacaggttgacgacaaacaggcctgtgacagcggggcttcgaactttccgtgtttttttttttcttccgctagtcgatcatccgtgacggccgggcaagcgcccgagtaccaggaccgtcgggcccccgagaagggggggcctaggatgcggcaagacgacccggttcggggaacgccagcgtgcgctggccgatcccgaggcgaggtgcc

SEQ ID NO: 7 - DNA, 208 bp, synthetic construct
  source 1..208: mol_type = other DNA
gaaacccaacagccgtgtatcgactcataggtttctaaccttaagaaatgaaattttcttaagtaagaaatactaggatagaagttttaaactataatatagcttctataacatgccaaggtacttaaaacttataagttttaagcacgagatatagtcagtgccattagagataatggaatagcatgtcccctcgcctccaccaata

SEQ ID NO: 8 - DNA, 119 bp, synthetic construct
  source 1..119: mol_type = other DNA
ctttcggacaggggttcgattcaaaagagtcgccttatgaagtgattcataagtgaaaacttggctttatcggtgaaaccgaaacgtaaagacgtcggcaataccgagtggtatgtggg

SEQ ID NO: 9 - DNA, 122 bp, synthetic construct
  source 1..122: mol_type = other DNA
gaaatggagaaggtgtagagactggaaggcaggtaccctaacagtaaagctgagggtaaagggacagtccagaccacaaactggttaatccaggcagtgaaaactgtagatggtaagcataa

SEQ ID NO: 10 - DNA, 129 bp, synthetic construct
  source 1..129: mol_type = other DNA
ctcaacgagtaagattgactaaacgcttaaaagttattcttactatgggcggtacgtaaagaaacttacgtatgtttacctgtcaaactcggggaagccaatagcgcggtaatcccgaaccaagctcca

SEQ ID NO: 11 - DNA, 117 bp, synthetic construct
  source 1..117: mol_type = other DNA
ccggggaccacgcgtgcagttcacagactagatgtcggtgggggatccgtctcctaagatatagtcgaggcccagcgcgaaagcctgggagtatccgcagataccgtcgtagtctta

SEQ ID NO: 12 - DNA, 274 bp, synthetic construct
  source 1..274: mol_type = other DNA
gttaggggatcgaagacgatttaaggaagcctcccggcctggtttccgtggaaatcaggccggagatggtatttggtcgtccaaagtaagcctgaaaggacttgctagtctcggccgtggccgaggcgacaccgtcaaattgcggggacctcctaaagcctggactaccaagccgacgccgaaaggcgccggtggccggggtaatgacctagggtatggtaacaacgtccgggatgtgacaatggacaatccgcagccaagcgctaccgcccct

SEQ ID NO: 13 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 14 - DNA, 558 bp, synthetic construct
  source 1..558: mol_type = other DNA
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 15 - DNA, 3816 bp, synthetic construct
  source 1..3816: mol_type = other DNA
atgttcgtgttcctggtgctgctgcctctggtgtccagccagtgtgtgaacctgcgcaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcggggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccgtcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatga

SEQ ID NO: 16 - DNA, 3636 bp, synthetic construct
  source 1..3636: mol_type = other DNA
aacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcatcgtgcgggagcccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgcctctgtgtacgcctggaaccggaagcggatcagcaattgcgtggccgactactccgtgctgtacaacctggccccattcttcaccttcaagtgctacggcgtgtcccctaccaagctgaacgacctgtgcttcacaaacgtgtacgccgacagcttcgtgatccggggagatgaagtgcggcagattgcccctggacagacaggcaatatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaaaccttgtaacggcgtggccggcttcaactgctacttcccactgcggtcctacagctttcggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaaaggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcttcagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 17 - DNA, 729 bp, synthetic construct
  source 1..729: mol_type = other DNA
ctaccgtttaatattgcgtcacgcgaaacgccaatatgctgaaaaacaaaaaacaaaaaacaaaaaaaccaaaaaacaaaacacaggtacccggggatccgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggaaaaaacaaaaaacaaaacagcatattgatgcgttacccagagatgttttcttgggt

SEQ ID NO: 18 - DNA, 1478 bp, synthetic construct
  source 1..1478: mol_type = other DNA
ctaccgtttaatattgcgtcacgcgaaacgccaatatgctgaaaaacaaaaaacaaaaaacaaaaaaaccaaaaaacaaaacacaggtacccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagcggccgcggggatccgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggaaaaaacaaaaaacaaaacagcatattgatgcgttacccagagatgttttcttgggt

SEQ ID NO: 19 - DNA, 755 bp, synthetic construct
  source 1..755: mol_type = other DNA
aaaatccgttgaccttaaacggtcgtgtgggttcaagtccctccacccccacgcgaaacgccaatatgctgaaaaacaaaaaacaaaaaacaaaaaaaccaaaaaacaaaacacaggtacccggggatccgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggaaaaaacaaaaaacaaaacagcatattgatgcgttaccagacgctacggactt

SEQ ID NO: 20 - DNA, 729 bp, synthetic construct
  source 1..729: mol_type = other DNA
ctgttatgcccttagatgttcgcgaaacgccaatatgctgaaaaacaaaaaacaaaaaacaaaaaaaccaaaaaacaaaacacaggtacccggggatccgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggaaaaaacaaaaaacaaaacagcatattgatgcgttaccagtttgaggcaataacaggt

SEQ ID NO: 21 - DNA, 729 bp, synthetic construct
  source 1..729: mol_type = other DNA
tcccctcgcctccaccaatacgcgaaacgccaatatgctgaaaaacaaaaaacaaaaaacaaaaaaaccaaaaaacaaaacacaggtacccggggatccgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggaaaaaacaaaaaacaaaacagcatattgatgcgttaccctttcggacaggggttcgat

SEQ ID NO: 22 - DNA, 708 bp, synthetic construct
  source 1..708: mol_type = other DNA
cataacgcgaaacgccaatatgctgaaaaacaaaaaacaaaaaacaaaaaaaccaaaaaacaaaacacagagctcggtacccggggatcctctagagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggaaaaaacaaaaaacaaaacagcatattgatgcgttaccct

SEQ ID NO: 23 - DNA, 741 bp, synthetic construct
  source 1..741: mol_type = other DNA
cagataccgtcgtagtcttacgcgaaacgccaatatgctgaaaaacaaaaaacaaaaaacaaaaaaaccaaaaaacaaaacacagagctcggtacccggggatcctctagagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggaaaaaacaaaaaacaaaacagcatattgatgcgttaccgttaggggatcgaagacgat

SEQ ID NO: 24 - DNA, 20 bp, synthetic construct
  source 1..20: mol_type = other DNA
ccgtcgattgtccactggtc

SEQ ID NO: 25 - DNA, 151 bp, synthetic construct
  source 1..151: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatgaagatagtagcgggccgggg

SEQ ID NO: 26 - DNA, 141 bp, synthetic construct
  source 1..141: mol_type = other DNA
accgaccagaatcatgcaagtgcttaaataattgagccttaaagaagaaattctttaagtggatgctctcaaactcagggaaacctaaatctagttatagacaaggcaatcctgagccaagccgaagtagtaattagtaag

SEQ ID NO: 27 - DNA, 70 bp, synthetic construct
  source 1..70: mol_type = other DNA
cgccggaaacgcaatagccgaaaaacaaaaaacaaaaaaaacaaaaaaaaaaccaaaaaaacaaaacaca

SEQ ID NO: 28 - DNA, 40 bp, synthetic construct
  source 1..40: mol_type = other DNA
aaaaaacaaaaaacaaaacggctattatgcgttaccggcg

(19) United States
(12) Patent Application Publication
(10) Pub. No.: US 2025/0243496 A1
(43) Pub. Date: Jul. 31, 2025

(54) VECTOR FOR GENERATING A CIRCULAR RNA

(71) Applicant: Agency for Science, Technology and Research, Singapore (SG)

(72) Inventors: Yue Wan, Singapore (SG); Kuo Chieh Liao, Singapore (SG); Xiang Gao, Singapore (SG)

(21) Appl. No.: 18/854,294

(22) PCT Filed: Apr. 6, 2023
(86) PCT No.: PCT/SG2023/050238

§ 371 Date: Oct. 4, 2024

(30) Foreign Application Priority Data
Apr. 6, 2022 (SG) 10202203526S

(51) Int. Cl. C12N 15/64; C07K 14/005; C12N 15/88

(52) U.S. Cl. CPC C12N 15/64; C07K 14/005; C12N 15/88; C12N 2770/20022; C12N 2770/24122; C12N 2840/203

(57) ABSTRACT

The invention relates to generally to the field of molecular biology. In particular, the invention is directed to a vector for generating a circular RNA. Methods for generating circular RNA from a precursor RNA transcribed from the vector are also provided herein.

### FIELD OF INVENTION

The invention relates to generally to the field of molecular biology. In particular, the invention is directed to a vector for generating a circular RNA. Methods for generating circular RNA from a precursor RNA transcribed from the vector are also provided herein.

### BACKGROUND

RNA therapy refers to the treatment or prevention of diseases using RNA-based molecules. There are a large number of RNA-based molecules that can be used for treating or vaccinating a subject. These includes messenger RNA (mRNA) molecules, which can produce a therapeutic protein, or express a viral protein to elicit an immune response. Other types of RNA-based molecules include antisense oligonucleotides (ASOs) or RNA interference (RNAi) molecules, which can be used to suppress the expression of specific genes in a patient. RNA molecules are also known to be unstable, and there is a constant need to improve the stability of RNA molecules for clinical development.

Current mRNA vaccines utilize a linear mRNA that is modified and capped, with a poly A tail. The RNA is then packaged with lipid nanoparticles and delivered into human cells through intramuscular injection. While highly effective, mRNA vaccine designs suffer from several drawbacks, including the need for low temperatures for transport and storage, the need for high doses to be injected, development of allergic reactions due to formulation, a lack of target specificity and high cost. As most of the mRNAs inside the cell are linear, degradation of the RNAs in mammalian cells occur predominantly through the activity of 5′ and 3′ exonucleases.

It would be desirable to overcome or ameliorate at least one of the above-described problems, or at least to provide a useful alternative.

### SUMMARY

Disclosed herein is a vector for generating a circular RNA, the vector comprising the following elements operably connected to each other and arranged in the following sequence: a) a 5′ Group I intron fragment, b) an internal ribosome entry site (IRES), c) a protein coding or noncoding region, and d) a 3′ Group I intron fragment.

Disclosed herein is a method of generating circular RNA from a precursor RNA transcribed from a vector as defined herein, the method comprising incubating the precursor RNA in the presence of a buffer and Mg²⁺ to generate circular RNA from the precursor RNA.

Disclosed herein is a circular RNA produced by a vector as defined herein.

Disclosed herein is a method of expressing protein in a cell, said method comprising transfecting the circular RNA as defined herein into the cell.

Disclosed herein is a nanoparticle composition comprising a circular RNA produced by a vector as defined herein.

Disclosed herein is a vector for generating a circular RNA, the vector comprising the following elements operably connected to each other and arranged in the following sequence: a) a 5′ homology sequence, b) an internal ribosome entry site (IRES), c) a protein coding or noncoding region, and d) a 3′ homology sequence.

Disclosed herein is a method of generating circular RNA from a precursor RNA transcribed from a vector as defined herein, the method comprising incubating the precursor RNA in the presence of a buffer and Mg²⁺ and a T4 RNA ligase I or II to generate circular RNA from the precursor RNA.

### BRIEF DESCRIPTION OF DRAWINGS

Embodiments of the present invention are hereafter described, by way of non-limiting example only, with reference to the accompanying drawings in which:

FIG. 1. Different ways of circularizing RNAs using split group 1 introns (left), T4 RNA ligase 1 (middle), T4 RNA ligase 2 (right). Exon sequences are retained in the circular RNA but that is only a small portion (<5%) as compared with the 5′ or 3′ intron fragments.

FIG. 2. Optimization of different features of the circular RNA design for efficient translation. Left, different group 1 introns, as well as different ligases for efficient RNA circularization, will be tested. Middle, different internal ribosomal entry sites from viral and human origins for efficient translation will be tested. Right, codon optimization will be performed and efficiently translated codon optimized sequences for the spike protein of SARS-COV-2 delta variant will be identified.

FIG. 3. Gel electrophoresis showing 0.5 kb insert or 1.5 kb insert RNA that are circularized with T4 RNA ligase 2 (Rnl2).

FIG. 4. Schematic representation of secondary structure of a Group I intron. Paired regions are numbered P1-P9 and the splice-sites are indicated with arrows, exons involved in structural interactions near the splice-sites are in dotted lines, and the GTP binding site is usually at P7 (G-OH). Permuted introns are created by making a cut at the loop region of conserved P6, the 5′-fragment is designated as E1 and the 3′-fragment is E2.

FIG. 5. Self-splicing efficiency of 1 kb construct of different group 1 introns. Each gel shows the self-splicing efficiency of a different group 1 intron under different buffer conditions (A-G). Input indicates the RNA before incubating in folding buffer to facilitate self-splicing. Green arrows indicate the circular product while the band on top is the linear product.

FIG. 6. Testing the accuracy of self-splicing of the different group 1 introns. Top, workflow of reverse transcription and PCR of circular RNA for Sanger sequencing. Bottom, table show the number of junctions in each category for each group I intron.

FIG. 7. Luciferase assay showing the amount of protein expression from the newly designed group 1 and T4 RNA ligase mediated circles are higher than existing group 1 introns (T4 and An).

FIG. 8. Circular version using CVB3 as internal ribosomal entry site has highest protein expression level. A panel of IRES were tested for protein expression level in Hela cells. Mock (no RNA) was included for control.

FIG. 9. A. Workflow of Circular RNA purification. B, Rnase R digestion showing digestion of linear products while preserving the circular RNAs.

FIG. 10. Workflow of generating circle RNA and encapsulating with polypeptide nanoparticles.

FIG. 11. Modified An PIE were folded in condition B and mixed with streptavidin CI beads. After washing and elution, bound fractions were analyzed by EX gel. These results show that circular RNA made from modified An (SEQ NO: 25 and NO: 26) can be purified by streptavidin beads.

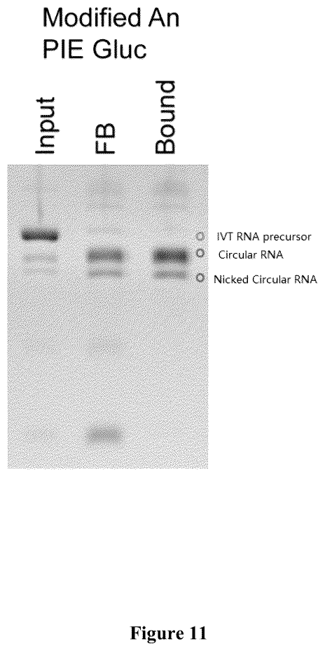

### DETAILED DESCRIPTION

The specification teaches a vector for generating a circular RNA. Disclosed herein is a vector for generating a circular RNA, the vector comprising the following elements operably connected to each other and arranged in the following sequence: a) a 5′ Group I intron fragment, b) an internal ribosome entry site (IRES), c) a protein coding or noncoding region, and d) a 3′ Group I intron fragment.

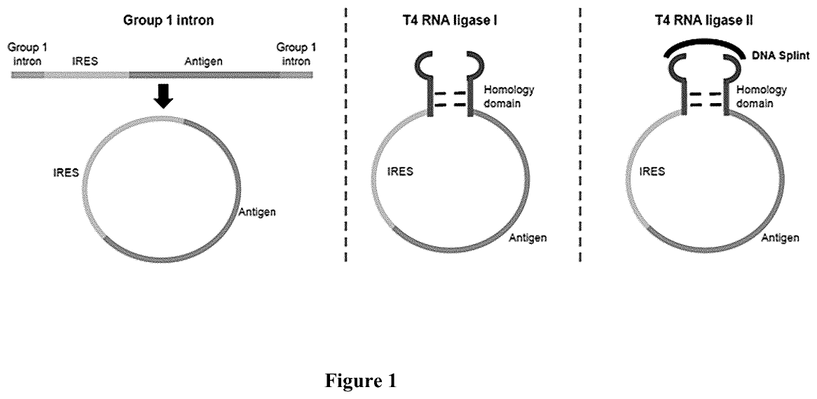

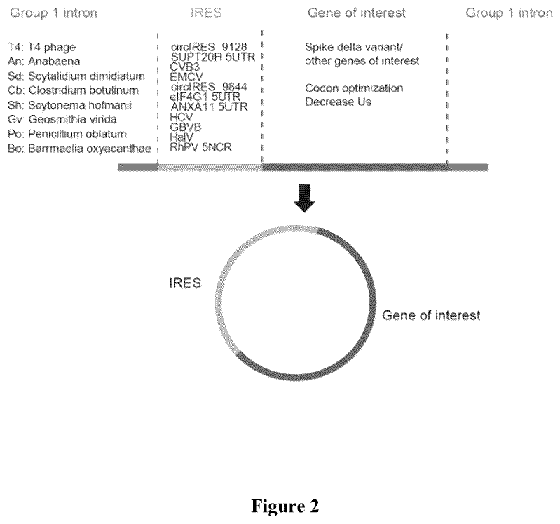

Without being bound by theory, the inventors have combined expertise in RNA biochemistry and RNA structural biology to develop a stable, cost-effective strategy for generating circular RNA vaccines with increased stability and translatability, which is broadly applicable to any pathogen of interest, including RNA viruses and pathogenic bacteria. The inventors have developed strategies to enable RNA circularization (FIG. 1) and protein translation from circular RNAs (FIG. 2). As these circular RNAs do not have free 5′ and 3′ ends that can be degraded by exonucleases, these endless RNA molecules can be highly stable inside cells, whereby their decay rates are across days, instead of hours. This property of circular RNAs enables it to serve as an expression platform for high and stable production of genes of interest. Additionally, while linear mRNAs require modifications including pseudo-uridylation to escape from the cellular immune system, circular RNAs do not need these modifications, reducing the cost of RNA production while maintaining scalability.

In one embodiment, the vector allows production of a circular RNA that is translatable and/or biologically active inside a cell, such as a eukaryotic cell.

As used herein, a “vector” means a piece of DNA, that is synthesized (e.g., using PCR), or that is taken from a virus, plasmid, or cell of a higher organism into which a foreign DNA fragment can be or has been inserted for cloning and/or expression purposes. In some embodiments, a vector can be stably maintained in an organism. A vector can comprise, for example, an origin of replication, a selectable marker or reporter gene, such as antibiotic resistance or GFP, and/or a multiple cloning site (MCS). The term includes linear DNA fragments (e.g., PCR products, linearized plasmid fragments), plasmid vectors, viral vectors, cosmids, bacterial artificial chromosomes (BACs), yeast artificial chromosomes (YACs), and the like.

As used herein, the elements of a vector are “operably connected” if they are positioned on the vector such that they can be transcribed to form a precursor RNA that can then be circularized into a circular RNA using the methods provided herein.

As used herein, the elements of a vector are “operably connected” if they are positioned on the vector such that they can be transcribed to form a precursor RNA that can then be circularized into a circular RNA using the methods provided herein.

As used herein, “precursor RNA” refers to a linear RNA molecule created by in vitro transcription (e.g., from a vector provided herein). This precursor RNA molecule contains the entirety of the circRNA sequence, plus splicing sequences (e.g. intron fragments or homology sequences) necessary to circularize the RNA. These splicing sequences (intron fragments or homology sequences) are substantially removed from the precursor RNA during circularization, yielding circRNA plus two intron/homology sequence linear RNA fragments. Precursor RNA can be unmodified, partially modified or completely modified. In one embodiment, the precursor RNA contains only naturally occurring nucleotides.

The terms “polynucleotide,” “polynucleotide sequence.” “nucleotide sequence.” “nucleic acid” or “nucleic acid sequence as used herein designate mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of RNA or DNA.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a poly A sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

As used herein, “modified” means non-natural. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

“Polypeptide.” “peptide,” “protein” and “proteinaceous molecule” are used interchangeably herein to refer to molecules comprising or consisting of a polymer of amino acid residues and to variants and synthetic analogues of the same.

As used herein, the term “encode” refers broadly to any process whereby the information in a polymeric macromolecule is used to direct the production of a second molecule that is different from the first. The second molecule may have a chemical structure that is different from the chemical nature of the first molecule.

In one embodiment, the 5′ and 3′ Group I intron fragments are from T4 phage, Cyanobacterium Anabaena, Scytalidium dimidiatum (Sd), Clostridium botulinum (Ch), Scytonema hofmanii (Sh), Geosmithia virida (Gv), Penicillium oblatum (Po) or Barrmaelia oxyacanthae (Bo). In one embodiment, the 5′ and 3′ Group I intron fragments are from Cyanobacterium Anabaena and Scytalidium dimidiatum (Sd),

In one embodiment, the S′ and 3′ Group I intron fragments are from Scytalidium dimidiatum (Sd), Clostridium botulinum (Ch), Scytonema hofmanii (Sh), Geosmithia virida (Gv), Penicillium oblatum (Po) or Barrmaelia oxyacanthae (Bo).

In one embodiment, the 5′ and 3′ Group I intron fragments are from Scytalidium dimidiatum (Sd), Clostridium botulinum (Cb), Scytonema hofmanii (Sh) or Penicillium oblatum (Po).

In one embodiment, the 5′ and 3′ Group I intron fragments are from Scytalidium dimidiatum (Sd).

As used herein, a 3′ group I intron fragment is a contiguous sequence that has at least 75% (e.g., at least 80%, at least 85%, at least 90%, at least 95%, 100%) sequence identity to a 3′ proximal fragment of a natural group I intron and, optionally, the adjacent exon sequence at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length). In one embodiment, the included adjacent exon sequence is about the length of the natural exon.

In some embodiments, a 5′ group I intron fragment is a contiguous sequence that has at least 75% (e.g., at least 80%, at least 85%, at least 90%, at least 95%, 100%) sequence identity to a 5′ proximal fragment of a natural group I intron and, optionally, the adjacent exon sequence at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length). In one embodiment, the included adjacent exon sequence is about the length of the natural exon.

In one embodiment, the 5′ Group I intron fragment comprises or consists of a nucleic acid sequence having at least 70% sequence identity to SEQ ID NO: 1, 3, 5, 7, 9 or 11. The 5′ Group I intron fragment may comprise or consist of a nucleic acid sequence having at least 70% sequence identity to SEQ ID NO: 5, 7, 9 or 11. The S′ Group I intron fragment may comprise or consist of a nucleic acid sequence having at least 70% sequence identity to SEQ ID NO: 5.

In one embodiment, the 3′ Group I intron fragment comprises or consists of a nucleic acid sequence having at least 70% sequence identity to SEQ ID NO: 2, 4, 6, 8, 10 or 12. The 3′ Group I intron fragment may comprise or consist of a nucleic acid sequence having at least 70% sequence identity to SEQ ID NO: 6, 8, 10 or 12. The 3′ Group I intron fragment may comprise or consist of a nucleic acid sequence having at least 70% sequence identity to SEQ ID NO: 6.

In another embodiment, the 5′ and 3′ Group I intron fragments are modified 5′ and 3′ Group I intron fragments from Cyanobacterium Anabaena. The 5′ Group I intron fragment may comprise or consist of a nucleic acid sequence having at least 70% sequence identity to SEQ ID NO: 25. The 3′ Group I intron fragment may comprise or consist of a nucleic acid sequence having at least 70% sequence identity to SEQ ID NO: 26.

The term “at least 70% sequence identity” may refer to at least 70%, 80%, 90%, 95%, 99% or 100% sequence identity.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Califomia, USA) using standard defaults as used in the reference manual accompanying the software.

Terms used to describe sequence relationships between two or more polynucleotides or polypeptides include “reference sequence,” “comparison window”, “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison. WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al . . . “Current Protocols in Molecular Biology”, John Wiley & Sons Inc. 1994-1998, Chapter 15.

In one embodiment, the vector further comprises at least one spacer domain. The at least one spacer domain may be positioned between the 5′ Group I intron fragment and the IRES.

As used herein, a “spacer” refers to a region of a polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex forming regions. The “spacer” may refer to any contiguous nucleotide sequence that is 1) predicted to avoid interfering with proximal structures, for example, from the IRES, coding or noncoding region, or intron 2) at least 7 nucleotides long (and optionally no longer than 100 nucleotides) 3) located downstream of and adjacent to the 3′ intron fragment and/or upstream of and adjacent to the 5′ intron fragment and/or 4) contains one or more of the following: a) an unstructured region at least 5nt long b) a region predicted base pairing at least 5nt long to a distal (i.e., non-adjacent) sequence, including another spacer, and/or c) a structured region at least 7nt long limited in scope to the sequence of the spacer.

In one embodiment, the spacer comprises a nucleic acid sequence having at least 70% sequence identity to SEQ ID NO: 27 and/or SEQ ID NO: 28.

As used herein, “interfering” with regard to sequences refers to sequence(s) predicted or empirically determined to alter the folding of other structures in the RNA, such as the IRES or group I intron-derived sequences.

As used herein, “unstructured” with regard to RNA refers to an RNA sequence that is not predicted by the RNAFold software or similar predictive tools to form a structure (e.g., a hairpin loop) with itself or other sequences in the same RNA molecule.

As used herein, “structured” with regard to RNA refers to an RNA sequence that is predicted by the RNAFold software or similar predictive tools to form a structure (e.g., a hairpin loop) with itself or other sequences in the same RNA molecule.

In some embodiments, the vector comprises an IRES sequence. The IRES sequence can be selected from, but not limited to, an IRES sequence of a Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stali intestine virus. Kashmir bee virus, Human rhinovirus 2, Homalodisca coagulata virus-1. Human Immunodeficiency Virus type 1, Homalodisca coagulata virus-1, Himetobi P virus. Hepatitis C virus, Hepatitis A virus, Hepatitis GB virus, foot and mouth disease virus, Human enterovirus 71. Equine rhinitis virus, Ectropis obliqua picorna-like virus, Encephalomyocarditis virus (EMCV), Drosophila C Virus. Crucifer tobamo virus. Cricket paralysis virus, Bovine viral diarrhea virus 1, Black Queen Cell Virus, Aphid lethal paralysis virus. Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human elF4G, Mouse NDST4L. Human LEF1, Mouse HIFI alpha, Human n.myc, Mouse Gtx, Human p27kip1, Human PDGF2/c-sis, Human p53, Human Pim-1. Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2). Wild-type IRES sequences can also be modified and be effective in the invention. In some embodiments, the IRES sequence is about 50 nucleotides in length.

In one embodiment, the IRES is an IRES sequence from viral CVB3, human SAT1, human HK1, viral RhPV, human elF4G1, viral HCV, viral HalIV or circIRES9128.

In one embodiment, the IRES is an IRES sequence from viral CVB3. The IRES may comprise or consist of a nucleic acid sequence having at least 70% sequence identity to SEQ ID NO: 13.

The vector as defined herein may comprise a protein coding or noncoding region.

In one embodiment, the vector comprises a protein coding region. The protein coding region can encode a protein for therapeutic use or diagnostic use. In some embodiments, the protein can be any protein for therapeutic use or diagnostic use. For example, the protein coding region can encode human protein or antibodies. In some embodiments, the protein can be selected from, but not limited to, hFIX, SP-B. VEGF-A, human methylmalonyl-CoA mutase (hMUT), CFTR, cancer self-antigens, and additional gene editing enzymes like Cpf1, zinc finger nucleases (ZFNs) and transcription activator-like effector nucleases (TALENs). In some embodiments, the protein can be a viral protein. The viral protein can be SARS-COV-2 spike protein or dengue EV71 protein.

In one embodiment, the protein coding region is more than 1 kb in size. The protein coding region may encode a viral protein (such as a SARS-COV-2 spike protein or dengue EV71 protein).

In some embodiments, the protein is a chimeric antigen receptor (CAR) or T cell receptor (TCR) complex protein. The CAR or TCR complex protein may comprise an antigen binding domain specific for a tumor antigen.

In some embodiments, the protein coding region encodes an mRNA sequence. The mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

In another embodiment, the vector comprises a noncoding region. In some embodiments, the noncoding regions can encode sequences that alter cellular behavior, such as e.g., lymphocyte behavior. In some embodiments, the noncoding sequences are antisense to cellular RNA sequences.

In some embodiments, the noncoding region encodes an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA is an immunomodulatory siRNA.

In some embodiments, the noncoding region encodes an shRNA. An shRNA may be produced inside a target cell upon delivery of an appropriate construct to the nucleus. Constructs and mechanisms relating to shRNA are well known in the relevant arts.

In one embodiment, the vector further comprises an RNA polymerase promoter. In another embodiment, the RNA polymerase promoter is a T7 virus RNA polymerase promoter, T6 virus RNA polymerase promoter, SP6 virus RNA polymerase promoter, T3 virus RNA polymerase promoter, or T4 virus RNA polymerase promoter.

In one embodiment, there is provided a precursor RNA comprising:
a) a 5′ Group I intron fragment,
b) an internal ribosome entry site (IRES),
c) a protein coding or noncoding region, and
d) a 3′ Group I intron fragment.

In one embodiment, the 5′ Group I intron fragment in the precursor RNA comprises or consists of a nucleic acid sequence having at least 70% sequence identity to a nucleic acid sequence encoded by any one of SEQ ID NO: 1, 3, 5, 7, 9 or 11

In one embodiment, the 3′ Group I intron fragment in the precursor RNA comprises or consists of a nucleic acid sequence having at least 70% sequence identity to a nucleic acid sequence encoded by any one of SEQ ID NO: 2, 4, 6, 8, 10 or 12.

Disclosed herein is a method of generating circular RNA from precursor RNA transcribed from a vector as defined herein, the method comprising incubating the precursor RNA in the presence of a buffer and Mg²⁺ generate circular RNA from the precursor RNA.

The buffer may be Hepes, Tris or other buffers. The temperature used maybe about 55° C. In one embodiment, the method comprises incubating the precursor RNA in the presence of 25 mM NaCl, 15 mM MgCl₂, 25 mM Hepes (pH7.5), 55° C.

The method may further comprises purifying the circular RNA.

Disclosed herein is a circular RNA produced by a vector as defined herein.

Disclosed herein is a method of expressing protein in a cell, said method comprising transfecting the circular RNA as defined herein into the cell.

Disclosed herein is a vector for generating a circular RNA, the vector comprising the following elements operably connected to each other and arranged in the following sequence: a) a 5′ homology sequence, b) an internal ribosome entry site (IRES), c) a protein coding or noncoding region, and d) a 3′ homology sequence.

As used herein, a “homology domain” or “homology sequence” is any contiguous sequence that is 1) predicted to form base pairs with at least about 75% (e.g., at least about 80%, at least about 85%, at least about 90%, at least about 95%, about 100%) of another sequence in the RNA, such as another homology arm 2) at least 7nt long and no longer than 250nt 3) located before and adjacent to, or included within, the 3′ intron fragment and/or after and adjacent to, or included within, the 5′ intron fragment and, optionally, 4) predicted to have less than 50% (e.g., less than 45%, less than 40%, less than 35%, less than 30%, less than 25%) base pairing with unintended sequences in the RNA (e.g., non-homology arm sequences). A “strong homology arm” refers to a homology arm with a Tm of greater than 50 degrees Celsius when base paired with another homology arm in the RNA.

Disclosed herein is a method of generating circular RNA from precursor RNA transcribed from a vector as defined herein, the method comprising incubating the precursor RNA in the presence of a buffer and a T4 RNA ligase I or II to generate circular RNA from the precursor RNA.

The buffer may be Hepes, Tris or other buffers. The temperature used maybe about 55° C. In one embodiment, the method comprises incubating the precursor RNA in the presence of 25 mM NaCl, 15 mM MgCl₂, 25 mM Hepes (pH7.5), 55° C.

Also provided here is a circular RNA as defined herein for use as a medicament.

Also provided here is a circular RNA as defined herein for use in treating a disease or a condition in a subject.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The terms “subject”, “patient”, “host” or “individual”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In one embodiment, the subject is a human subject.

The present specification also discloses nanoparticle compositions comprising a circular RNA as described herein.

As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more mRNA to one or more target cells.

Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

As used herein, “transfer vehicle” includes any of the standard pharmaceutical carriers, diluents, excipients, and the like, which are generally intended for use in connection with the administration of biologically active agents, including nucleic acids.

In some embodiments, the invention also relates to compositions, e.g., compositions comprising a circular RNA and a pharmaceutically acceptable carrier. Pharmaceutical compositions of the present disclosure may comprise a circular RNA as described herein, in combination with one or more pharmaceutically or physiologically acceptable carriers, excipients or diluents. In some embodiments, pharmaceutical compositions of the present disclosure may comprise a circular RNA expressing cell as described herein, in combination with one or more pharmaceutically or physiologically acceptable carriers, excipients or diluents.

In some embodiments, a pharmaceutically acceptable carrier can be an ingredient in a pharmaceutical composition, other than an active ingredient, which is nontoxic to the subject.

A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, saccharides, antioxidants, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

In some embodiments, such compositions may comprise buffers such as acetic acid, citric acid, histidine, boric acid, formic acid, succinic acid, phosphoric acid, carbonic acid, malic acid, aspartic acid, Tris buffers, HEPPSO, HEPES, neutral buffered saline, phosphate buffered saline and the like; carbohydrates such as glucose, sucrose, mannose, or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

In certain embodiments, compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

In one embodiment, there is provided a method of preventing or treating a disease or condition in a subject, the method comprising administering an effective amount of a vector, a circular RNA, nanoparticle composition or pharmaceutical composition as described herein to the subject.

In one embodiment, the disease or condition is a viral infection such as coronavirus (e.g. SARS-CoV-2) infection or dengue infection. In another embodiment, the disease or condition is a cancer.

The term “administering” refers to contacting, applying, injecting, transfusing or providing a drug as referred to herein to a subject.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

In some embodiments, a circular RNA as described herein may be used in combination with other known agents and therapies. Administered “in combination”, as used herein, means that two (or more) different treatments are delivered to the subject during the course of the subject's treatment e.g., the two or more treatments are delivered after the subject has been diagnosed with the disease and before the disease has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In further embodiments, a composition described herein may be used in a treatment regimen in combination with surgery, radiation, chemotherapy, antibodies, or other agents.

As used herein, “and/or” refers to and encompasses any and all possible combinations of one or more of the associated listed items, as well as the lack of combinations when interpreted in the alternative (or).

As used in this application, the singular form “a,” “an.” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Circularization of RNA Using T4 RNA Ligase II can be Optimized Using Different Conditions.

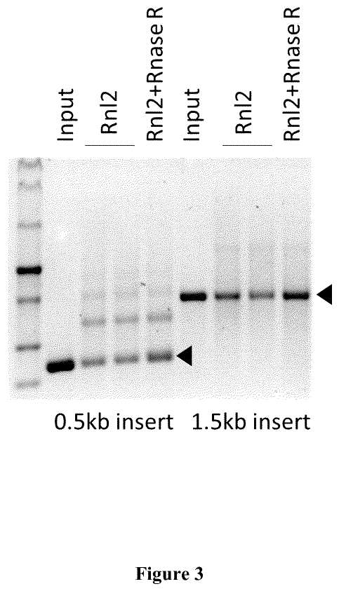

Different homology sequences (such as from AnCS20 and dengue (DVCS26)) to bring the 5′ and 3′ ends of the RNA close together, different treatment conditions to enable the ends to be ligation compatible, and different ligation enzyme and times to identify the conditions that facilitate the most efficient circularization, were tested. It was found that using T4 RNA ligase 2 and overnight ligation produces ˜53% circularized RNA for a 1 kb long RNA (FIG. 3).

Design of Self-Circularizing RNAs Using Permuted Group 1 Introns from Natural-Existing Ribozymes.

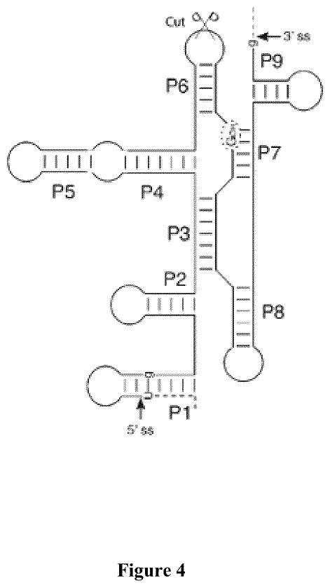

Group 1 introns are a class of self-catalytic RNAs that can splice its own introns and exist in different organisms including T4 phage (T4) and Anabaena (An). All group I introns share regions of conserved structure that build up the core ribozyme region (FIG. 4), the conserved secondary structure for group 1 introns includes 9 paired regions numbered P1-P9 and the splice-sites (5′-ss and 3′-ss) are indicated with arrows. All group I introns contain these paired regions except for P2. The permuted group I intron could be created by cutting at P6 without affecting the 5′- and 3′-splicing activity. Attaching the permuted ends of a group I intron to the termini of a linear RNA enables back-splicing, which results in self-catalytic circularization.

Identification of New Group 1 Introns that can be Used to Generate Circular RNAs Using Split Intron-Exon Strategy.

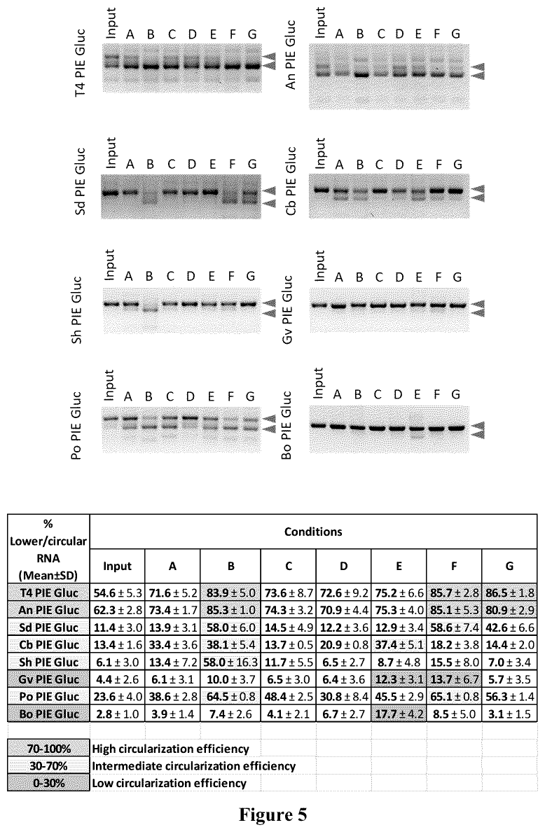

Several members group 1 introns such that those from T4 phage and from Anabaena has been used to generate circular RNAs with high efficiencies by self-splicing. However, most group 1 introns only work well with short inserts of a few hundred bases. As many of the genes of interest are long (for e.g, spike protein is 4 kb, and VP1-3 proteins from EV71 is 2.5 kb), group 1 introns from different organisms were tested for their self-splicing efficiencies using inserts of different lengths. In addition to T4 and An, whose group I introns are known to be able to circularize RNA using split intron-exon strategy, 6 different group I introns (Scytalidium dimidiatum (Sd), Clostridium botulinum (Cb), Scytonema hofmanii (Sh), Geosmithia virida (Gv), Penicillium oblatum (Po) and Barrmaelia oxyacanthae (Bo)) were designed and tested for their ability to self-splice, and hence circularize the RNA, in the presence of Gluc as insert (FIG. 5). 7 different folding conditions (A-G) for self-splicing efficiency of the different group I introns were also tested as each intron could require different folding conditions for self-splicing.

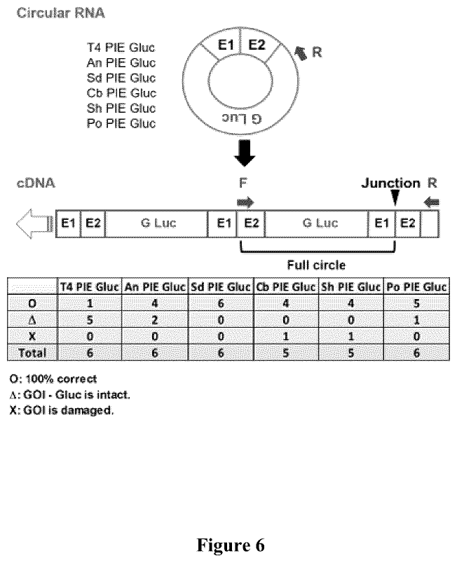

As self-splicing of RNA can sometimes introduce errors such as insertions and deletions between the splice sites, the accuracy of self-splicing for these group 1 introns was checked by performing reverse transcription, PCR followed by Sanger sequencing on the circular RNAs. The self-spliced junctions were classified into three categories: 1) completely accurate, 2) some insertions and deletions that do not affect the genes of interest, 3) errors that disrupt the genes of interest and hence will not express the genes of interest correctly. It was observed that Sd contains the most number of highly accurate junctions, followed by Po. An and T4 (FIG. 6), suggesting that they could act as good candidates for circularization of RNA.

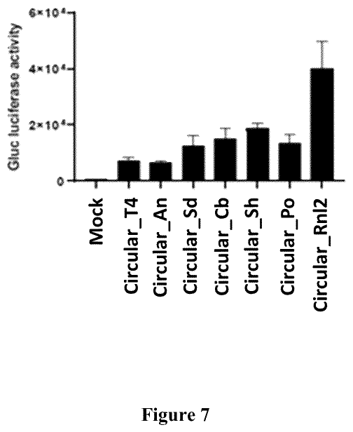

Newly designed group 1 introns and T4 RNA ligase 2 can outperform existing group I introns in protein production (FIG. 7).

### Identification of IRES for High Circular RNA Translation

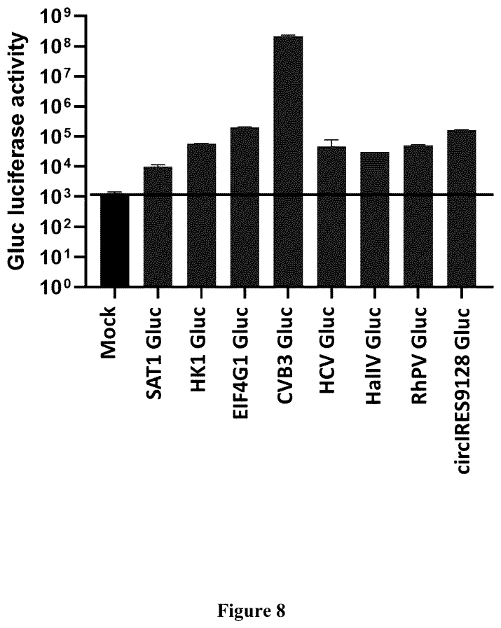

Unlike the cap-dependent translation mechanism in mRNA, the endless circular RNA uses IRES (internal ribosome entry sequence) for cap-independent protein translation. A panel of IRES from different viral and human origins were tested to identify IRES that can drive strong translation (FIG. 8). Top candidates could be tested with different GOI (gene of interest) for translation efficiency in different cellular environments.

### Strategies for Circular RNA Purification, Up-Scaling and QC.

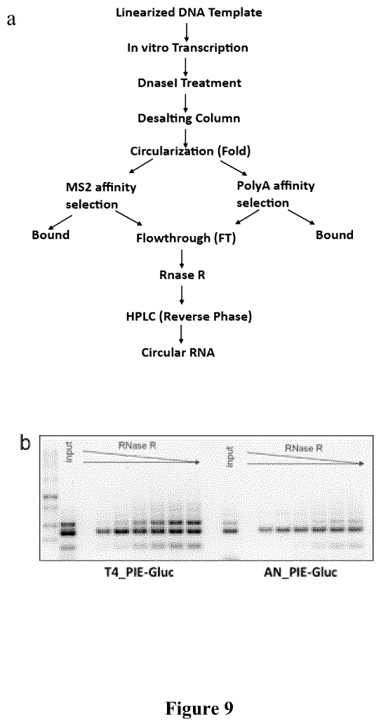

As a part of the nucleic acid manufacturing procedure, developing an effective downstream bioprocess for the enrichment of pure circular RNA is essential. Therefore, a general production pipeline was developed specifically for circular RNAs (FIG. 9a). Briefly, linear RNA containing the permuted group I intron is transcribed from a linearized DNA template in vitro, and then folded and back-spliced using conditions from FIG. 3, the linear/circular RNA mixture is then treated with a specific concentration of RNase R to remove majority of linear RNAs (FIG. 9b).

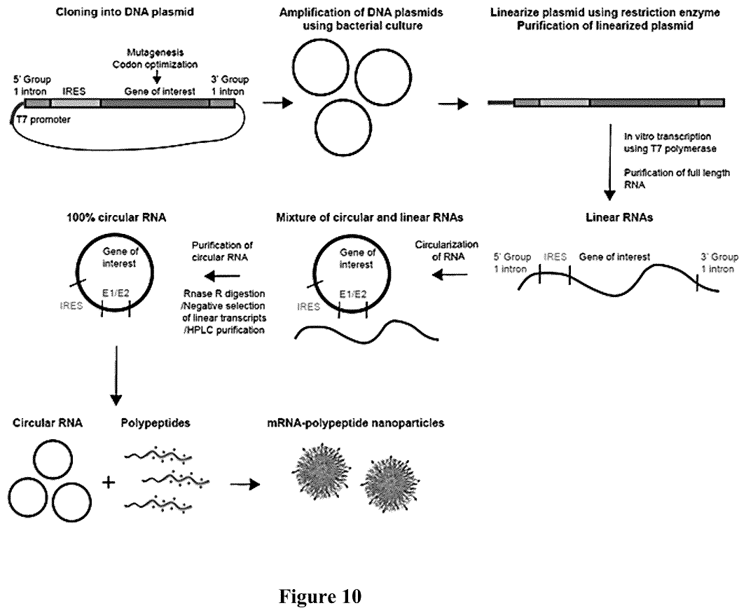

The workflow for generating circle RNAs follows standard mRNA manufacturing practices and is manufactured in RNA foundries to generate GMP grade RNAs and nanoparticles for vaccine production and clinical trials. The steps in making the circular RNA are outlined in detail (FIG. 10). Briefly, a DNA plasmid containing permuted group I introns, IRES and gene of interest is amplified using bacteria culture, the plasmid is purified and linearized using restriction enzyme digestion, next followed by in vitro transcription using RNA polymerase and the linear RNAs are then circularized under specific folding conditions. Finally, the circular RNAs are purified using chromatographic technique described in FIG. 9. QC will be performed on the circular RNAs by reverse transcription, library preparation and deep sequencing. Only circular RNAs with a high proportion of accurate junctions will be used in the downstream packaging.

| | Sequence |
|---|---|
| T4 5′ Group 1 Intron | AATGGAATTGGTTCTACATAAATGCCTAACGACTA |
| fragment | TCCCTTTGGGGAGTAGGGTCAAGTGACTCGAAAC |
| | GATAGACAACTTGCTTTAACAAGTTGGAGATATA |
| | GTCTGCTCTGCATGGTGACATGCAGCTGGATATAA |
| | TTCCGGGGTAAGATTAACGACCTTATCTGAACATA |
| | ATGCTACCGTTTAATATTGCGTCA (SEQ ID NO: 1) |
| T4 3′ Group 1 Intron | CAGAGATGTTTTCTTGGGTTAATTGAGGCCTGAGT |
| fragment | ATAAGGTGACTTATACTTGTAATCTATCTAAACGG |
| | GGAACCTCTCTAGTAGACAATCCCGTGCTAAATTG |
| | TAGGACT (SEQ ID NO: 2) |
| An 5′ Group 1 Intron | AACAATAGATGACTTACAACTAATCGGAAGGTGC |
| fragment | AGAGACTCGACGGGAGCTACCCTAACGTCAAGAC |
| | GAGGGTAAAGAGAGAGTCCAATTCTCAAAGCCAA |
| | TAGGCAGTAGCGAAAGCTGCAAGAGAATGAAAAT |
| | CCGTTGACCTTAAACGGTCGTGTGGGTTCAAGTCC |
| | CTCCACCCCCA (SEQ ID NO: 3) |
| An 3′ Group 1 Intron | AGACGCTACGGACTTAAATAATTGAGCCTTAAAG |
| fragment | AAGAAATTCTTTAAGTGGATGCTCTCAAACTCAGG |
| | GAAACCTAAATCTAGTTATAGACAAGGCAATCCT |
| | GAGCCAAGCCGAAGTAGTAATTAGTAAG (SEQ ID |
| | NO: 4) |
| Sd 5′ Group 1 Intron | GTAGCGGGCACCCGTCGTAACGCGCGGTAGGGCG |
| fragment | TCGGTCCCCCCTCCCACGGCGGAGGGGAGGCTTA |
| | AGGTACGTGCTAAACCCCCAGCGATGGGGCCTGT |
| | AGGAAAAGCCCTGGTACGGCGAAGCCTACGGGGA |
| | CCGCCCGATGGCGGTCGGATGCCGGGGGCCACG |
| | GAGCCCCCGGCGTCGATTGCTGTTATGCCCTTAGA |
| | TGTT (SEQ ID NO: 5) |
| Sd 3′ Group 1 Intron | AGTTTGAGGCAATAACAGGTTGACGACAAACAGG |
| fragment | CCTGTGACAGCGGGGCTTCGAACTTTCCGTGTTTT |
| | TTTTTTTCTTCCGCTAGTCGATCATCCGTGACGGCC |
| | GGGCAAGCGCCCGAGTACCAGGACCGTCGGGCCC |
| | CCGAGAAGGGGGGGCCTAGGATGCGGCAAGACG |
| | ACCCGGTTCGGGGAACGCCAGCGTGCGCTGGCCG |
| | ATCCCGAGGCGAGGTGCC (SEQ ID NO: 6) |
| Cb 5′ Group 1 Intron | GAAACCCAACAGCCGTGTATCGACTCATAGGTTTC |
| fragment | TAACCTTAAGAAATGAAATTTTCTTAAGTAAGAAA |
| | TACTAGGATAGAAGTTTTAAACTATAATATAGCTT |
| | CTATAACATGCCAAGGTACTTAAAACTTATAAGTT |
| | TTAAGCACGAGATATAGTCAGTGCCATTAGAGAT |
| | AATGGAATAGCATGTCCCCTCGCCTCCACCAATA |
| | (SEQ ID NO: 7) |
| Cb 3′ Group 1 Intron | CTTTCGGACAGGGGTTCGATTCAAAAGAGTCGCCT |
| fragment | TATGAAGTGATTCATAAGTGAAAACTTGGCTTTAT |
| | CGGTGAAACCGAAACGTAAAGACGTCGGCAATAC |
| | CGAGTGGTATGTGGG (SEQ ID NO: 8) |
| Sh 5′ Group 1 Intron | GAAATGGAGAAGGTGTAGAGACTGGAAGGCAGGT |
| fragment | ACCCTAACAGTAAAGCTGAGGGTAAAGGGACAGT |
| | CCAGACCACAAACTGGTTAATCCAGGCAGTGAAA |
| | ACTGTAGATGGTAAGCATAA (SEQ ID NO: 9) |
| Sh 3′ Group 1 Intron | CTCAACGAGTAAGATTGACTAAACGCTTAAAAGT |
| fragment | TATTCTTACTATGGGGGGTACGTAAAGAAACTTAC |
| | GTATGTTTACCTGTCAAACTCGGGGAAGCCAATAG |
| | CGCGGTAATCCCGAACCAAGCTCCA (SEQ ID NO: |
| | 10) |
| Po 5′ Group 1 Intron | CCGGGGACCACGCGTGCAGTTCACAGACTAGATG |
| fragment | TCGGTGGGGGATCCGTCTCCTAAGATATAGTCGAG |
| | GCCCAGCGCGAAAGCCTGGGAGTATCCGCAGATA |
| | CCGTCGTAGTCTTA (SEQ ID NO: 11) |
| Po 3′ Group 1 Intron | GTTAGGGGATCGAAGACGATTTAAGGAAGCCTCC |
| fragment | CGGCCTGGTTTCCGTGGAAATCAGGCCGGAGATG |
| | GTATTTGGTCGTCCAAAGTAAGCCTGAAAGGACTT |
| | GCTAGTCTCGGCCGTGGCCGAGGCGACACCGTCA |
| | AATTGCGGGGACCTCCTAAAGCCTGGACTACCAA |
| | GCCGACGCCGAAAGGCGCCGGTGGCCGGGGTAAT |
| | GACCTAGGGTATGGTAACAACGTCCGGGATGTGA |
| | CAATGGACAATCCGCAGCCAAGCGCTACCGCCCC |
| | T (SEQ ID NO: 12) |
| CVB3 IRES | TTAAAACAGCCTGTGGGTTGATCCCACCCACAGGC |
| | CCATTGGGCGCTAGCACTCTGGTATCACGGTACCT |
| | TTGTGCGCCTGTTTTATACCCCCTCCCCCAACTGTA |
| | ACTTAGAAGTAACACACACCGATCAACAGTCAGC |
| | GTGGCACACCAGCCACGTTTTGATCAAGCACTTCT |
| | GTTACCCCGGACTGAGTATCAATAGACTGCTCACG |
| | CGGTTGAAGGAGAAAGCGTTCGTTATCCGGCCAA |
| | CTACTTCGAAAAACCTAGTAACACCGTGGAAGTT |
| | GCAGAGTGTTTCGCTCAGCACTACCCCAGTGTAGA |
| | TCAGGTCGATGAGTCACCGCATTCCCCACGGGCG |
| | ACCGTGGCGGTGGCTGCGTTGGCGGCCTGCCCATG |
| | GGGAAACCCATGGGACGCTCTAATACAGACATGG |
| | TGCGAAGAGTCTATTGAGCTAGTTGGTAGTCCTCC |
| | GGCCCCTGAATGCGGCTAATCCTAACTGCGGAGC |
| | ACACACCCTCAAGCCAGAGGGCAGTGTGTCGTAA |
| | CGGGCAACTCTGCAGCGGAACCGACTACTTTGGG |
| | TGTCCGTGTTTCATTTTATTCCTATACTGGCTGCTT |
| | ATGGTGACAATTGAGAGATCGTTACCATATAGCTA |
| | TTGGATTGGCCATCCGGTGACTAATAGAGCTATTA |
| | TATATCCCTTTGTTGGGTTTATACCACTTAGCTTGA |
| | AAGAGGTTAAAACATTACAATTCATTGTTAAGTTG |
| | AATACAGCAAA (SEQ ID NO: 13) |
| Gaussia Luciferase | ATGGGAGTCAAAGTTCTGTTTGCCCTGATCTGCAT |
| | CGCTGTGGCCGAGGCCAAGCCCACCGAGAACAAC |
| | GAAGACTTCAACATCGTGGCCGTGGCCAGCAACT |
| | TCGCGACCACGGATCTCGATGCTGACCGCGGGAA |
| | GTTGCCCGGCAAGAAGCTGCCGCTGGAGGTGCTC |
| | AAAGAGATGGAAGCCAATGCCCGGAAAGCTGGCT |
| | GCACCAGGGGCTGTCTGATCTGCCTGTCCCACATC |
| | AAGTGCACGCCCAAGATGAAGAAGTTCATCCCAG |
| | GACGCTGCCACACCTACGAAGGCGACAAAGAGTC |
| | CGCACAGGGGGGCATAGGCGAGGCGATCGTCGAC |
| | ATTCCTGAGATTCCTGGGTTCAAGGACTTGGAGCC |
| | CATGGAGCAGTTCATCGCACAGGTCGATCTGTGTG |
| | TGGACTGCACAACTGGCTGCCTCAAAGGGCTTGCC |
| | AACGTGCAGTGTTCTGACCTGCTCAAGAAGTGGCT |
| | GCCGCAACGCTGTGCGACCTTTGCCAGCAAGATCC |
| | AGGGCCAGGTGGACAAGATCAAGGGGGCCGGTGG |
| | TGACTAA (SEQ ID NO: 14) |
| SARS-CoV2 Delta | ATGTTCGTGTTCCTGGTGCTGCTGCCTCTGGTGTCC |
| spike | AGCCAGTGTGTGAACCTGCGCACCAGAACACAGC |
| | TGCCTCCAGCCTACACCAACAGCTTTACCAGAGGC |
| | GTGTACTACCCCGACAAGGTGTTCAGATCCAGCGT |
| | GCTGCACTCTACCCAGGACCTGTTCCTGCCTTTCTT |
| | CAGCAACGTGACCTGGTTCCACGCCATCCACGTGT |
| | CCGGCACCAATGGCACCAAGAGATTCGACAACCC |
| | CGTGCTGCCCTTCAACGACGGGGTGTACTTTGCCA |
| | GCACCGAGAAGTCCAACATCATCAGAGGCTGGAT |
| | CTTCGGCACCACACTGGACAGCAAGACCCAGAGC |
| | CTGCTGATCGTGAACAACGCCACCAACGTGGTCAT |
| | CAAAGTGTGCGAGTTCCAGTTCTGCAACGACCCCT |
| | TCCTGGACGTCTACTACCACAAGAACAACAAGAG |
| | CTGGATGGAAAGCGGGGTGTACAGCAGCGCCAAC |
| | AACTGCACCTTCGAGTACGTGTCCCAGCCTTTCCT |
| | GATGGACCTGGAAGGCAAGCAGGGCAACTTCAAG |
| | AACCTGCGCGAGTTCGTGTTTAAGAACATCGACG |
| | GCTACTTCAAGATCTACAGCAAGCACACCCCTATC |
| | AACCTCGTGCGGGATCTGCCTCAGGGCTTCTCTGC |
| | TCTGGAACCCCTGGTGGATCTGCCCATCGGCATCA |
| | ACATCACCCGGTTTCAGACACTGCTGGCCCTGCAC |
| | AGAAGCTACCTGACACCTGGCGATAGCAGCAGCG |
| | GATGGACAGCTGGTGCCGCCGCTTACTATGTGGGC |
| | TACCTGCAGCCTAGAACCTTCCTGCTGAAGTACAA |
| | CGAGAACGGCACCATCACCGACGCCGTGGATTGT |
| | GCTCTGGATCCTCTGAGCGAGACAAAGTGCACCCT |
| | GAAGTCCTTCACCGTGGAAAAGGGCATCTACCAG |
| | ACCAGCAACTTCCGGGTGCAGCCCACCGAATCCA |
| | TCGTGCGGTTCCCCAATATCACCAATCTGTGCCCC |
| | TTCGGCGAGGTGTTCAATGCCACCAGATTCGCCTC |
| | TGTGTACGCCTGGAACCGGAAGCGGATCAGCAAT |
| | TGCGTGGCCGACTACTCCGTGCTGTACAACTCCGC |
| | CAGCTTCAGCACCTTCAAGTGCTACGGCGTGTCCC |
| | CTACCAAGCTGAACGACCTGTGCTTCACAAACGTG |
| | TACGCCGACAGCTTCGTGATCCGGGGAGATGAAG |
| | TGCGGCAGATTGCCCCTGGACAGACAGGCAAGAT |
| | CGCCGACTACAACTACAAGCTGCCCGACGACTTC |
| | ACCGGCTGTGTGATTGCCTGGAACAGCAACAACC |
| | TGGACTCCAAAGTCGGCGGCAACTACAATTACCG |
| | GTACCGGCTGTTCCGGAAGTCCAATCTGAAGCCCT |
| | TCGAGCGGGACATCTCCACCGAGATCTATCAGGC |
| | CGGCAGCAAGCCTTGTAACGGCGTGGAAGGCTTC |
| | AACTGCTACTTCCCACTGCAGTCCTACGGCTTTCA |
| | GCCCACAAATGGCGTGGGCTATCAGCCCTACAGA |
| | GTGGTGGTGCTGAGCTTCGAACTGCTGCATGCCCC |
| | TGCCACAGTGTGCGGCCCTAAGAAAAGCACCAAT |
| | CTCGTGAAGAACAAATGCGTGAACTTCAACTTCA |
| | ACGGCCTGACCGGCACCGGCGTGCTGACAGAGAG |
| | CAACAAGAAGTTCCTGCCATTCCAGCAGTTTGGCC |
| | GGGATATCGCCGATACCACAGACGCCGTTAGAGA |
| | TCCCCAGACACTGGAAATCCTGGACATCACCCCTT |
| | GCAGCTTCGGCGGAGTGTCTGTGATCACCCCTGGC |
| | ACCAACACCAGCAATCAGGTGGCAGTGCTGTACC |
| | AGGGCGTGAACTGTACCGAAGTGCCCGTGGCCAT |
| | TCACGCCGATCAGCTGACACCTACATGGCGGGTGT |
| | ACTCCACCGGCAGCAATGTGTTTCAGACCAGAGC |
| | CGGCTGTCTGATCGGAGCCGAGCACGTGAACAAT |
| | AGCTACGAGTGCGACATCCCCATCGGCGCTGGAA |
| | TCTGCGCCAGCTACCAGACACAGACAAACAGCCG |
| | TCGGAGAGCCAGAAGCGTGGCCAGCCAGAGCATC |
| | ATTGCCTACACAATGTCTCTGGGCGCCGAGAACA |
| | GCGTGGCCTACTCCAACAACTCTATCGCTATCCCC |
| | ACCAACTTCACCATCAGCGTGACCACAGAGATCCT |
| | GCCTGTGTCCATGACCAAGACCAGCGTGGACTGC |
| | ACCATGTACATCTGCGGCGATTCCACCGAGTGCTC |
| | CAACCTGCTGCTGCAGTACGGCAGCTTCTGCACCC |
| | AGCTGAATAGAGCCCTGACAGGGATCGCCGTGGA |
| | ACAGGACAAGAACACCCAAGAGGTGTTCGCCCAA |
| | GTGAAGCAGATCTACAAGACCCCTCCTATCAAGG |
| | ACTTCGGCGGCTTCAATTTCAGCCAGATTCTGCCC |
| | GATCCTAGCAAGCCCAGCAAGCGGAGCTTCATCG |
| | AGGACCTGCTGTTCAACAAAGTGACACTGGCCGA |
| | CGCCGGCTTCATCAAGCAGTATGGCGATTGTCTGG |
| | GCGACATTGCCGCCAGGGATCTGATTTGCGCCCAG |
| | AAGTTTAACGGACTGACAGTGCTGCCTCCTCTGCT |
| | GACCGATGAGATGATCGCCCAGTACACATCTGCC |
| | CTGCTGGCCGGCACAATCACAAGCGGCTGGACAT |
| | TTGGAGCAGGCGCCGCTCTGCAGATCCCCTTTGCT |
| | ATGCAGATGGCCTACCGGTTCAACGGCATCGGAG |
| | TGACCCAGAATGTGCTGTACGAGAACCAGAAGCT |
| | GATCGCCAACCAGTTCAACAGCGCCATCGGCAAG |
| | ATCCAGGACAGCCTGAGCAGCACAGCAAGCGCCC |
| | TGGGAAAGCTGCAGAACGTGGTCAACCAGAATGC |
| | CCAGGCACTGAACACCCTGGTCAAGCAGCTGTCCT |
| | CCAACTTCGGCGCCATCAGCTCTGTGCTGAACGAT |
| | ATCCTGAGCAGACTGGACCCTCCTGAGGCCGAGG |
| | TGCAGATCGACAGACTGATCACAGGCAGACTGCA |
| | GAGCCTCCAGACATACGTGACCCAGCAGCTGATC |
| | AGAGCCGCCGAGATTAGAGCCTCTGCCAATCTGG |
| | CCGCCACCAAGATGTCTGAGTGTGTGCTGGGCCA |
| | GAGCAAGAGAGTGGACTTTTGCGGCAAGGGCTAC |
| | CACCTGATGAGCTTCCCTCAGTCTGCCCCTCACGG |
| | CGTGGTGTTTCTGCACGTGACATATGTGCCCGCTC |
| | AAGAGAAGAATTTCACCACCGCTCCAGCCATCTG |
| | CCACGACGGCAAAGCCCACTTTCCTAGAGAAGGC |
| | GTGTTCGTGTCCAACGGCACCCATTGGTTCGTGAC |
| | ACAGCGGAACTTCTACGAGCCCCAGATCATCACC |
| | ACCGACAACACCTTCGTGTCTGGCAACTGCGACGT |
| | CGTGATCGGCATTGTGAACAATACCGTGTACGACC |
| | CTCTGCAGCCCGAGCTGGACAGCTTCAAAGAGGA |
| | ACTGGACAAGTACTTTAAGAACCACACAAGCCCC |
| | GACGTGGACCTGGGCGATATCAGCGGAATCAATG |
| | CCAGCGTCGTGAACATCCAGAAAGAGATCGACCG |
| | GCTGAACGAGGTGGCCAAGAATCTGAACGAGAGC |
| | CTGATCGACCTGCAAGAACTGGGGAAGTACGAGC |
| | AGTACATCAAGTGGCCCTGGTACATCTGGCTGGGC |
| | TTTATCGCCGGACTGATTGCCATCGTGATGGTCAC |
| | AATCATGCTGTGTTGCATGACCAGCTGCTGTAGCT |
| | GCCTGAAGGGCTGTTGTAGCTGTGGCAGCTGCTGC |
| | AAGTTCGACGAGGACGATTCTGAGCCCGTGCTGA |
| | AGGGCGTGAAACTGCACTACACATGA (SEQ ID NO: |
| | 15) |
| SARS-CoV2 Omicron | AACGTGACCTGGTTCCACGTGATCTCCGGCACCAA |
| Spike | TGGCACCAAGAGATTCGACAACCCCGTGCTGCCCT |
| | TCAACGACGGGGTGTACTTTGCCAGCATCGAGAA |
| | GTCCAACATCATCAGAGGCTGGATCTTCGGCACCA |
| | CACTGGACAGCAAGACCCAGAGCCTGCTGATCGT |
| | GAACAACGCCACCAACGTGGTCATCAAAGTGTGC |
| | GAGTTCCAGTTCTGCAACGACCCCTTCCTGGACCA |
| | CAAGAACAACAAGAGCTGGATGGAAAGCGAGTTC |
| | CGGGTGTACAGCAGCGCCAACAACTGCACCTTCG |
| | AGTACGTGTCCCAGCCTTTCCTGATGGACCTGGAA |
| | GGCAAGCAGGGCAACTTCAAGAACCTGCGCGAGT |
| | TCGTGTTTAAGAACATCGACGGCTACTTCAAGATC |
| | TACAGCAAGCACACCCCTATCATCGTGCGGGAGC |
| | CCGAAGATCTGCCTCAGGGCTTCTCTGCTCTGGAA |
| | CCCCTGGTGGATCTGCCCATCGGCATCAACATCAC |
| | CCGGTTTCAGACACTGCTGGCCCTGCACAGAAGCT |
| | ACCTGACACCTGGCGATAGCAGCAGCGGATGGAC |
| | AGCTGGTGCCGCCGCTTACTATGTGGGCTACCTGC |
| | AGCCTAGAACCTTCCTGCTGAAGTACAACGAGAA |
| | CGGCACCATCACCGACGCCGTGGATTGTGCTCTGG |
| | ATCCTCTGAGCGAGACAAAGTGCACCCTGAAGTC |
| | CTTCACCGTGGAAAAGGGCATCTACCAGACCAGC |
| | AACTTCCGGGTGCAGCCCACCGAATCCATCGTGCG |
| | GTTCCCCAATATCACCAATCTGTGCCCCTTCGATG |
| | AGGTGTTCAATGCCACCAGATTCGCCTCTGTGTAC |
| | GCCTGGAACCGGAAGCGGATCAGCAATTGCGTGG |
| | CCGACTACTCCGTGCTGTACAACCTGGCCCCATTC |
| | TTCACCTTCAAGTGCTACGGCGTGTCCCCTACCAA |
| | GCTGAACGACCTGTGCTTCACAAACGTGTACGCCG |
| | ACAGCTTCGTGATCCGGGGAGATGAAGTGCGGCA |
| | GATTGCCCCTGGACAGACAGGCAATATCGCCGAC |
| | TACAACTACAAGCTGCCCGACGACTTCACCGGCTG |
| | TGTGATTGCCTGGAACAGCAACAAGCTGGACTCC |
| | AAAGTCAGCGGCAACTACAATTACCTGTACCGGC |
| | TGTTCCGGAAGTCCAATCTGAAGCCCTTCGAGCGG |
| | GACATCTCCACCGAGATCTATCAGGCCGGCAACA |
| | AACCTTGTAACGGCGTGGCCGGCTTCAACTGCTAC |
| | TTCCCACTGCGGTCCTACAGCTTTCGGCCCACATA |
| | CGGCGTGGGCCACCAGCCCTACAGAGTGGTGGTG |
| | CTGAGCTTCGAACTGCTGCATGCCCCTGCCACAGT |
| | GTGCGGCCCTAAGAAAAGCACCAATCTCGTGAAG |
| | AACAAATGCGTGAACTTCAACTTCAACGGCCTGA |
| | AAGGCACCGGCGTGCTGACAGAGAGCAACAAGAA |
| | GTTCCTGCCATTCCAGCAGTTTGGCCGGGATATCG |
| | CCGATACCACAGACGCCGTTAGAGATCCCCAGAC |
| | ACTGGAAATCCTGGACATCACCCCTTGCAGCTTCG |
| | GCGGAGTGTCTGTGATCACCCCTGGCACCAACACC |
| | AGCAATCAGGTGGCAGTGCTGTACCAGGGCGTGA |
| | ACTGTACCGAAGTGCCCGTGGCCATTCACGCCGAT |
| | CAGCTGACACCTACATGGCGGGTGTACTCCACCG |
| | GCAGCAATGTGTTTCAGACCAGAGCCGGCTGTCTG |
| | ATCGGAGCCGAGTACGTGAACAATAGCTACGAGT |
| | GCGACATCCCCATCGGCGCTGGAATCTGCGCCAG |
| | CTACCAGACACAGACAAAGAGCCACCGGAGAGCC |
| | AGAAGCGTGGCCAGCCAGAGCATCATTGCCTACA |
| | CAATGTCTCTGGGCGCCGAGAACAGCGTGGCCTA |
| | CTCCAACAACTCTATCGCTATCCCCACCAACTTCA |
| | CCATCAGCGTGACCACAGAGATCCTGCCTGTGTCC |
| | ATGACCAAGACCAGCGTGGACTGCACCATGTACA |
| | TCTGCGGCGATTCCACCGAGTGCTCCAACCTGCTG |
| | CTGCAGTACGGCAGCTTCTGCACCCAGCTGAAGA |
| | GAGCCCTGACAGGGATCGCCGTGGAACAGGACAA |
| | GAACACCCAAGAGGTGTTCGCCCAAGTGAAGCAG |
| | ATCTACAAGACCCCTCCTATCAAGTACTTCGGCGG |
| | CTTCAATTTCAGCCAGATTCTGCCCGATCCTAGCA |
| | AGCCCAGCAAGCGGAGCTTCATCGAGGACCTGCT |
| | GTTCAACAAAGTGACACTGGCCGACGCCGGCTTC |
| | ATCAAGCAGTATGGCGATTGTCTGGGCGACATTGC |
| | CGCCAGGGATCTGATTTGCGCCCAGAAGTTTAAG |
| | GGACTGACAGTGCTGCCTCCTCTGCTGACCGATGA |
| | GATGATCGCCCAGTACACATCTGCCCTGCTGGCCG |
| | GCACAATCACAAGCGGCTGGACATTTGGAGCAGG |
| | CGCCGCTCTGCAGATCCCCTTTGCTATGCAGATGG |
| | CCTACCGGTTCAACGGCATCGGAGTGACCCAGAA |
| | TGTGCTGTACGAGAACCAGAAGCTGATCGCCAAC |
| | CAGTTCAACAGCGCCATCGGCAAGATCCAGGACA |
| | GCCTGAGCAGCACAGCAAGCGCCCTGGGAAAGCT |
| | GCAGGACGTGGTCAACCACAATGCCCAGGCACTG |
| | AACACCCTGGTCAAGCAGCTGTCCTCCAAGTTCGG |
| | CGCCATCAGCTCTGTGCTGAACGATATCTTCAGCA |
| | GACTGGACCCTCCTGAGGCCGAGGTGCAGATCGA |
| | CAGACTGATCACAGGCAGACTGCAGAGCCTCCAG |
| | ACATACGTGACCCAGCAGCTGATCAGAGCCGCCG |
| | AGATTAGAGCCTCTGCCAATCTGGCCGCCACCAA |
| | GATGTCTGAGTGTGTGCTGGGCCAGAGCAAGAGA |
| | GTGGACTTTTGCGGCAAGGGCTACCACCTGATGA |
| | GCTTCCCTCAGTCTGCCCCTCACGGCGTGGTGTTT |
| | CTGCACGTGACATATGTGCCCGCTCAAGAGAAGA |
| | ATTTCACCACCGCTCCAGCCATCTGCCACGACGGC |
| | AAAGCCCACTTTCCTAGAGAAGGCGTGTTCGTGTC |
| | CAACGGCACCCATTGGTTCGTGACACAGCGGAAC |
| | TTCTACGAGCCCCAGATCATCACCACCGACAACAC |
| | CTTCGTGTCTGGCAACTGCGACGTCGTGATCGGCA |
| | TTGTGAACAATACCGTGTACGACCCTCTGCAGCCC |
| | GAGCTGGACAGCTTCAAAGAGGAACTGGACAAGT |
| | ACTTTAAGAACCACACAAGCCCCGACGTGGACCT |
| | GGGCGATATCAGCGGAATCAATGCCAGCGTCGTG |
| | AACATCCAGAAAGAGATCGACCGGCTGAACGAGG |
| | TGGCCAAGAATCTGAACGAGAGCCTGATCGACCT |
| | GCAAGAACTGGGGAAGTACGAGCAGTACATCAAG |
| | TGGCCCTGGTACATCTGGCTGGGCTTTATCGCCGG |
| | ACTGATTGCCATCGTGATGGTCACAATCATGCTGT |
| | GTTGCATGACCAGCTGCTGTAGCTGCCTGAAGGGC |
| | TGTTGTAGCTGTGGCAGCTGCTGCAAGTTCGACGA |
| | GGACGATTCTGAGCCCGTGCTGAAGGGCGTGAAA |
| | CTGCACTACACATGATGA (SEQ ID NO: 16) |
| T4 circular RNA with | CTACCGTTTAATATTGCGTCACGCGAAACGCCAAT |
| Gaussia Luciferase | ATGCTGAAAAACAAAAAACAAAAAACAAAAAAA |
| | CCAAAAAAAAAACACAGGTACCCGGGGATCOGC |
| | CACCATGGGAGTCAAAGTTCTGTTTGCCCTGATCT |
| | GCATCGCTGTGGCCGAGGCCAAGCCCACCGAGAA |
| | CAACGAAGACTTCAACATCGTGGCCGTGGCCAGC |
| | AACTTCGCGACCACGGATCTCGATGCTGACCGCG |
| | GGAAGTTGCCCGGCAAGAAGCTGCCGCTGGAGGT |
| | GCTCAAAGAGATGGAAGCCAATGCCCGGAAAGCT |
| | GGCTGCACCAGGGGCTGTCTGATCTGCCTGTCCCA |
| | CATCAAGTGCACGCCCAAGATGAAGAAGTTCATC |
| | CCAGGACGCTGCCACACCTACGAAGGCGACAAAG |
| | AGTCCGCACAGGGCGGCATAGGCGAGGCGATOGT |
| | CGACATTCCTGAGATTCCTGGGTTCAAGGACTTGG |
| | AGCCCATGGAGCAGTTCATCGCACAGGTCGATCT |
| | GTGTGTGGACTGCACAACTGGCTGCCTCAAAGGG |
| | CTTGCCAACGTGCAGTGTTCTGACCTGCTCAAGAA |
| | GTGGCTGCCGCAACGCTGTGCGACCTTTGCCAGCA |
| | AGATCCAGGGCCAGGTGGACAAGATCAAGGGGGC |
| | CGGTGGTGACTAACCTGCAGGAAAAAACAAAAAA |
| | CAAAACAGCATATTGATGCGTTACCCAGAGATGTT |
| | TTCTTGGGT (SEQ ID NO: 17) |
| T4 circular RNA with | CTACCGTTTAATATTGCGTCACGCGAAACGCCAAT |
| CVB3 and Gaussia | ATGCTGAAAAACAAAAAACAAAAAACAAAAAAA |
| Luciferase | CCAAAAAACAAAACACAGGTACCCTTAAAACAGC |
| | CTGTGGGTTGATCCCACCCACAGGCCCATTGGGCG |
| | CTAGCACTCTGGTATCACGGTACCTTTGTGCGCCT |
| | GTTTTATACCCCCTCCCCCAACTGTAACTTAGAAG |
| | TAACACACACCGATCAACAGTCAGCGTGGCACAC |
| | CAGCCACGTTTTGATCAAGCACTTCTGTTACCCCG |
| | GACTGAGTATCAATAGACTGCTCACGCGGTTGAA |
| | GGAGAAAGCGTTCGTTATCCGGCCAACTACTTCGA |
| | AAAACCTAGTAACACCGTGGAAGTTGCAGAGTGT |
| | TTCGCTCAGCACTACCCCAGTGTAGATCAGGTCGA |
| | TGAGTCACCGCATTCCCCACGGGCGACCGTGGCG |
| | GTGGCTGCGTTGGCGGCCTGCCCATGGGGAAACC |
| | CATGGGACGCTCTAATACAGACATGGTGCGAAGA |
| | GTCTATTGAGCTAGTTGGTAGTCCTCCGGCCCCTG |
| | AATGCGGCTAATCCTAACTGCGGAGCACACACCC |
| | TCAAGCCAGAGGGCAGTGTGTCGTAACGGGCAAC |
| | TCTGCAGCGGAACCGACTACTTTGGGTGTCCGTGT |
| | TTCATTTTATTCCTATACTGGCTGCTTATGGTGACA |
| | ATTGAGAGATCGTTACCATATAGCTATTGGATTGG |
| | CCATCCGGTGACTAATAGAGCTATTATATATCCCT |
| | TTGTTGGGTTTATACCACTTAGCTTGAAAGAGGTT |
| | AAAACATTACAATTCATTGTTAAGTTGAATACAGC |
| | AAAGCGGCCGCGGGGATCCGCCACCATGGGAGTC |
| | AAAGTTCTGTTTGCCCTGATCTGCATCGCTGTGGC |
| | CGAGGCCAAGCCCACCGAGAACAACGAAGACTTC |
| | AACATCGTGGCCGTGGCCAGCAACTTCGCGACCA |
| | CGGATCTCGATGCTGACCGCGGGAAGTTGCCCGG |
| | CAAGAAGCTGCCGCTGGAGGTGCTCAAAGAGATG |
| | GAAGCCAATGCCCGGAAAGCTGGCTGCACCAGGG |
| | GCTGTCTGATCTGCCTGTCCCACATCAAGTGCACG |
| | CCCAAGATGAAGAAGTTCATCCCAGGACGCTGCC |
| | ACACCTACGAAGGCGACAAAGAGTCCGCACAGGG |
| | CGGCATAGGCGAGGCGATCGTCGACATTCCTGAG |
| | ATTCCTGGGTTCAAGGACTTGGAGCCCATGGAGC |
| | AGTTCATCGCACAGGTCGATCTGTGTGTGGACTGC |
| | ACAACTGGCTGCCTCAAAGGGCTTGCCAACGTGC |
| | AGTGTTCTGACCTGCTCAAGAAGTGGCTGCCGCAA |
| | CGCTGTGCGACCTTTGCCAGCAAGATCCAGGGCC |
| | AGGTGGACAAGATCAAGGGGGCCGGTGGTGACTA |
| | ACCTGCAGGAAAAAACAAAAAACAAAACAGCAT |
| | ATTGATGCGTTACCCAGAGATGTTTTCTTGGGT |
| | (SEQ ID NO: 18) |
| An circular RNA with | AAAATCCGTTGACCTTAAACGGTCGTGTGGGTTCA |
| Gaussia Luciferase | AGTCCCTCCACCCCCACGCGAAACGCCAATATGCT |
| | GAAAAACAAAAAACAAAAAACAAAAAAACCAAA |
| | AAACAAAACACAGGTACCCGGGGATCCGCCACCA |
| | TGGGAGTCAAAGTTCTGTTTGCCCTGATCTGCATC |
| | GCTGTGGCCGAGGCCAAGCCCACCGAGAACAACG |
| | AAGACTTCAACATCGTGGCCGTGGCCAGCAACTTC |
| | GCGACCACGGATCTCGATGCTGACCGCGGGAAGT |
| | TGCCCGGCAAGAAGCTGCCGCTGGAGGTGCTCAA |
| | AGAGATGGAAGCCAATGCCCGGAAAGCTGGCTGC |
| | ACCAGGGGCTGTCTGATCTGCCTGTCCCACATCAA |
| | GTGCACGCCCAAGATGAAGAAGTTCATCCCAGGA |
| | CGCTGCCACACCTACGAAGGCGACAAAGAGTCCG |
| | CACAGGGCGGCATAGGCGAGGCGATCGTCGACAT |
| | TCCTGAGATTCCTGGGTTCAAGGACTTGGAGCCCA |
| | TGGAGCAGTTCATCGCACAGGTCGATCTGTGTGTG |
| | GACTGCACAACTGGCTGCCTCAAAGGGCTTGCCA |
| | ACGTGCAGTGTTCTGACCTGCTCAAGAAGTGGCTG |
| | CCGCAACGCTGTGCGACCTTTGCCAGCAAGATCCA |
| | GGGCCAGGTGGACAAGATCAAGGGGGCCGGTGGT |
| | GACTAACCTGCAGGAAAAAACAAAAAACAAAAC |
| | AGCATATTGATGCGTTACCAGACGCTACGGACTT |
| | (SEQ ID NO: 19) |
| Sd circular RNA with | CTGTTATGCCCTTAGATGTTCGCGAAACGCCAATA |
| Gaussia Luciferase | TGCTGAAAAACAAAAAACAAAAAACAAAAAAAC |
| | CAAAAAACAAAACACAGGTACCCGGGGATCCGCC |
| | ACCATGGGAGTCAAAGTTCTGTTTGCCCTGATCTG |
| | CATCGCTGTGGCCGAGGCCAAGCCCACCGAGAAC |
| | AACGAAGACTTCAACATCGTGGCCGTGGCCAGCA |
| | ACTTCGCGACCACGGATCTCGATGCTGACCGCGG |
| | GAAGTTGCCCGGCAAGAAGCTGCCGCTGGAGGTG |
| | CTCAAAGAGATGGAAGCCAATGCCCGGAAAGCTG |
| | GCTGCACCAGGGGCTGTCTGATCTGCCTGTCCCAC |
| | ATCAAGTGCACGCCCAAGATGAAGAAGTTCATCC |
| | CAGGACGCTGCCACACCTACGAAGGCGACAAAGA |
| | GTCCGCACAGGGCGGCATAGGCGAGGCGATCGTC |
| | GACATTCCTGAGATTCCTGGGTTCAAGGACTTGGA |
| | GCCCATGGAGCAGTTCATCGCACAGGTCGATCTGT |
| | GTGTGGACTGCACAACTGGCTGCCTCAAAGGGCTT |
| | GCCAACGTGCAGTGTTCTGACCTGCTCAAGAAGTG |
| | GCTGCCGCAACGCTGTGCGACCTTTGCCAGCAAG |
| | ATCCAGGGCCAGGTGGACAAGATCAAGGGGGCCG |
| | GTGGTGACTAACCTGCAGGAAAAAACAAAAAACA |
| | AAACAGCATATTGATGCGTTACCAGTTTGAGGCA |
| | ATAACAGGT (SEQ ID NO: 20) |
| Cb circular RNA with | TCCCCTCGCCTCCACCAATACGCGAAACGCCAATA |
| Gaussia Luciferase | TGCTGAAAAACAAAAAACAAAAAACAAAAAAAC |
| | CAAAAAACAAAACACAGGTACCCGGGGATCCGCC |
| | ACCATGGGAGTCAAAGTTCTGTTTGCCCTGATCTG |
| | CATCGCTGTGGCCGAGGCCAAGCCCACCGAGAAC |
| | AACGAAGACTTCAACATCGTGGCCGTGGCCAGCA |
| | ACTTCGCGACCACGGATCTCGATGCTGACCGCGG |
| | GAAGTTGCCCGGCAAGAAGCTGCCGCTGGAGGTG |
| | CTCAAAGAGATGGAAGCCAATGCCCGGAAAGCTG |
| | GCTGCACCAGGGGCTGTCTGATCTGCCTGTCCCAC |
| | ATCAAGTGCACGCCCAAGATGAAGAAGTTCATCC |
| | CAGGACGCTGCCACACCTACGAAGGCGACAAAGA |
| | GTCCGCACAGGGCGGCATAGGCGAGGCGATCGTC |
| | GACATTCCTGAGATTCCTGGGTTCAAGGACTTGGA |
| | GCCCATGGAGCAGTTCATCGCACAGGTCGATCTGT |
| | GTGTGGACTGCACAACTGGCTGCCTCAAAGGGCTT |
| | GCCAACGTGCAGTGTTCTGACCTGCTCAAGAAGTG |
| | GCTGCCGCAACGCTGTGCGACCTTTGCCAGCAAG |
| | ATCCAGGGCCAGGTGGACAAGATCAAGGGGGCCG |
| | GTGGTGACTAACCTGCAGGAAAAAACAAAAAACA |
| | AAACAGCATATTGATGCGTTACCCTTTCGGACAGG |
| | GGTTCGAT (SEQ ID NO: 21) |
| Sh circular RNA with | CATAACGCGAAACGCCAATATGCTGAAAAACAAA |
| Gaussia Luciferase | AAACAAAAAACAAAAAAACCAAAAAACAAAACA |
| | CAGAGCTCGGTACCCGGGGATCCTCTAGAGCCAC |
| | CATGGGAGTCAAAGTTCTGTTTGCCCTGATCTGCA |
| | TCGCTGTGGCCGAGGCCAAGCCCACCGAGAACAA |
| | CGAAGACTTCAACATCGTGGCCGTGGCCAGCAAC |
| | TTCGCGACCACGGATCTCGATGCTGACCGOGGGA |
| | AGTTGCCCGGCAAGAAGCTGCCGCTGGAGGTGCT |
| | CAAAGAGATGGAAGCCAATGCCCGGAAAGCTGGC |
| | TGCACCAGGGGCTGTCTGATCTGCCTGTCCCACAT |
| | CAAGTGCACGCCCAAGATGAAGAAGTTCATCCCA |
| | GGACGCTGCCACACCTACGAAGGCGACAAAGAGT |
| | CCGCACAGGGGGGCATAGGCGAGGCGATCGTCGA |
| | CATTCCTGAGATTCCTGGGTTCAAGGACTTGGAGC |
| | CCATGGAGCAGTTCATCGCACAGGTCGATCTGTGT |
| | GTGGACTGCACAACTGGCTGCCTCAAAGGGCTTG |
| | CCAACGTGCAGTGTTCTGACCTGCTCAAGAAGTGG |
| | CTGCCGCAACGCTGTGCGACCTTTGCCAGCAAGAT |
| | CCAGGGCCAGGTGGACAAGATCAAGGGGGCCGGT |
| | GGTGACTAACCTGCAGGAAAAAACAAAAAACAAA |
| | ACAGCATATTGATGCGTTACCCT (SEQ ID NO: 22) |
| Po circular RNA with | CAGATACCGTCGTAGTCTTACGCGAAACGCCAAT |
| Gaussia Luciferase | ATGCTGAAAAACAAAAAACAAAAAACAAAAAAA |
| | CCAAAAAAAAAACACAGAGCTCGGTACCCGGGG |
| | ATCCTCTAGAGCCACCATGGGAGTCAAAGTTCTGT |
| | TTGCCCTGATCTGCATCGCTGTGGCCGAGGCCAAG |
| | CCCACCGAGAACAACGAAGACTTCAACATCGTGG |
| | CCGTGGCCAGCAACTTCGCGACCACGGATCTCGAT |
| | GCTGACCGCGGGAAGTTGCCCGGCAAGAAGCTGC |
| | CGCTGGAGGTGCTCAAAGAGATGGAAGCCAATGC |
| | CCGGAAAGCTGGCTGCACCAGGGGCTGTCTGATC |
| | TGCCTGTCCCACATCAAGTGCACGCCCAAGATGA |
| | AGAAGTTCATCCCAGGACGCTGCCACACCTACGA |
| | AGGCGACAAAGAGTCCGCACAGGGCGGCATAGGC |
| | GAGGCGATCGTCGACATTCCTGAGATTCCTGGGTT |
| | CAAGGACTTGGAGCCCATGGAGCAGTTCATCGCA |
| | CAGGTCGATCTGTGTGTGGACTGCACAACTGGCTG |
| | CCTCAAAGGGCTTGCCAACGTGCAGTGTTCTGACC |
| | TGCTCAAGAAGTGGCTGCCGCAACGCTGTGCGAC |
| | CTTTGCCAGCAAGATCCAGGGCCAGGTGGACAAG |
| | ATCAAGGGGGCCGGTGGTGACTAACCTGCAGGAA |
| | AAAACAAAAAACAAAACAGCATATTGATGCGTTA |
| | CCGTTAGGGGATCGAAGACGAT (SEQ ID NO: 23) |
| AnCS20 homology arm | CCGTCGATTGTCCACTGGTC (SEQ ID NO: 24) |
| Modified NO3-AN 5′ | AACAATAGATGACTTACAACTAATCGGAAGGTGC |
| Group 1 intron | AGAGACTCGACGGGAGCTACCCTAACGTCAAGAC |
| sequence: | GAGGGTAAAGAGAGAGTCCAATTCTCAAAGCCAA |
| | TAGGCAGTAGCGAAAGCTGCAAGAGAATG |
| | AAGATAGT AGC GGG C CGGGG (SEQ ID NO: 25) |
| Modified NO4 AN 3′ | ACCGACCAGAATCA TGC AAGT GCT T |
| Group 1 intron | AAATAATTGAGCCTTAAAGAAGAAATTCTTTAAGT |
| sequence: | GGATGCTCTCAAACTCAGGGAAACCTAAATCTAG |
| | TTATAGACAAGGCAATCCTGAGCCAAGCCGAAGT |
| | AGTAATTAGTAAG (SEQ ID NO: 26) |
| Spacer 1 | CGCCGGAAACGCAATAGCCGAAAAACAAAAAAC |
| | AAAAAAAACAAAAAAAAAACCAAAAAAACAAAA |
| | CACA (SEQ ID NO: 27) |
| Spacer 2 | AAAAAACAAAAAACAAAACGGCTATTATGCGTTA |
| | CCGGCG (SEQ ID NO: 28) |

1. A vector for generating a circular RNA, the vector comprising the following elements operably connected to each other and arranged in the following sequence:
a) a 5′ Group I intron fragment,
b) an internal ribosome entry site (IRES),
c) a protein coding or noncoding region, and
d) a 3′ Group I intron fragment.

2. The vector of claim 1, wherein the vector further comprises at least one spacer domain.

3. The vector of claim 1 wherein the 5′ and 3′ Group I intron fragments are from Scytalidium dimidiatum (Sd), Clostridium botulinum (Cb), Scytonema hofmanii (Sh), Geosmithia virida (Gv), Penicillium oblatum (Po) or Barrmaelia oxyacanthae (Bo).

4. The vector of claim 1, wherein the 5′ Group I intron fragment comprise or consist of a nucleic acid sequence having at least 70% sequence identity to SEQ ID NO: 5 and the 3′ Group I intron fragment comprise or consist of a nucleic acid sequence having at least 70% sequence identity to SEQ ID NO: 6.

5. The vector of claim 1, wherein the 5′ Group I intron fragment comprise or consist of a nucleic acid sequence having at least 70% sequence identity to SEQ ID NO: 25 and the 3′ Group I intron fragment comprise or consist of a nucleic acid sequence having at least 70% sequence identity to SEQ ID NO: 26.

6. The vector of claim 1, wherein the IRES is an IRES sequence from viral CVB3, human SAT1, human HK1, viral RhPV, human eIF4G1, viral HCV, viral HalIV or circIRES9128.

7. The vector of claim 1, wherein the protein coding region is more than 1 kb in size.

8. The vector of claim 1, wherein the protein coding region encodes an mRNA.

9. The vector of claim 1, wherein the protein coding region encodes a viral protein.

10. The vector of claim 1, wherein the vector further comprises an RNA polymerase positioned upstream of the 5′ Group I intron fragment.

11. The vector of claim 10, wherein the RNA polymerase promoter is an RNA polymerase promotor from T7 virus, T6 virus, SP6 virus, T3 virus, or T4 virus.

12. A method of generating circular RNA from a precursor RNA transcribed from a vector of claim 1, the method comprising incubating the precursor RNA in the presence of a buffer and Mg²⁺ to generate circular RNA from the precursor RNA.

13. The method of claim 12, wherein the method further comprises purifying the circular RNA.

14. A circular RNA produced by a vector of claim 1.

15. A method of expressing protein in a cell, said method comprising transfecting the circular RNA of claim 14 into the cell.

16. A nanoparticle composition comprising a circular RNA produced by a vector of claim 1.

17.-18. (canceled)
